(19) United States
(12) Patent Application Publication
(10) Pub. No.: US 2024/0407690 A1
(43) Pub. Date: Dec. 12, 2024

(54) SYSTEMS FOR THE COLLECTION AND INTRA-OPERATIVE ANALYSIS OF BIOLOGICAL TISSUES

(71) Applicant: Beams, Paris (FR)

(72) Inventors: Bruno Boyer, Palaiseau (FR); Laurent Menard, Palaiseau (FR); Stéphane Palfi, Le Val-Saint-Germain (FR); Virginie Simon, Vineui (FR); Estelle Villedieu De Torcy, Bagneux (FR)

(21) Appl. No.: 18/737,466

(22) Filed: Jun. 7, 2024

(30) Foreign Application Priority Data
Jun. 9, 2023 (FR) 2305857

(51) Int. Cl. A61B 5/20; A61B 10/00; G01T 1/20

(52) U.S. Cl. CPC A61B 5/207; A61B 10/007; G01T 1/2018

(57) ABSTRACT

A system for sampling biological tissue from a zone of interest receiving a biomarker prior to or simultaneously with surgery includes an excision head connected to a receptacle for the tissue samples by a suction transfer tube, a detection sleeve surrounding a reference zone of the suction transfer pipe, the detection sleeve comprising at least one sensor sensitive to the biomarker, and an electronic circuit delivering an electrical signal representative of the presence or absence of a marker when a tissue sample passes through the reference zone.

### PRIORITY CLAIM

This application claims the benefit of the filing date of French Patent Application Serial No. FR2305857, filed Jun. 9, 2023, for “Systems for Intraoperative Collection and Analysis of Biological Tissue.”

### TECHNICAL FIELD

The present disclosure relates to surgical equipment for sampling any biological tissue, in particular, tumors, using a detection and/or excision head connected to a suction tube for transferring the sampled tissue to a container that receives the sampled tissue for real-time analysis as it moves through the suction tube and/or for subsequent anatomopathological diagnosis, including proteomic, metabolomic or genotypic studies.

### BACKGROUND

To facilitate localization of the tissues to be detected and analyzed, a product is injected intraoperatively either in the vicinity of the areas of interest, or intravascularly or lymphatically. Three products are most often used: a dye (injected in the operating room a few minutes before surgery), a fluorescent molecule or a radioactive product (injected a few hours before surgery or intraoperatively). These products are sometimes combined.

Collected tissue is detected, counted and geolocated in the tumor region, then stored in identified and labeled containers. The collected tissues are then either analyzed in the operating room or sent to the laboratory for immediate pathologicalanalysis, or possibly biochemical and genotyping tests.

These techniques can also be used to improve the accuracy of tumor diagnosis from biopsies by guiding the surgeon to relevant tissue regions to determine the histological nature of the tumor.

These techniques make the surgical procedure safer by providing the surgeon with real-time information on the tumoral or non-tumoral nature of tissue samples.

In the prior art, U.S. Patent Application Publication No. 2012/283563 A1 relates to a biopsy system comprising a needle, a cutter movable relative to the needle to sever a tissue sample, a processing module, a tissue sensor, and an indicator. The tissue sensor detects the passage of a tissue sample cut by the cutter. The indicator emits an audible signal and/or a visual indicator. The indication provided by the indicator may vary based on sensed qualities of the tissue sample. The indicator may be integrated into a biopsy instrument or may be provided as part of a remote unit. The biopsy system may also comprise a multi-chamber tissue sample holder. The graphical user interface may indicate which chambers of the tissue sample holder are occupied by tissue samples.

This solution is used to check whether or not a sample has been captured.

U.S. Patent Application Publication No. 2016/100852 A1 describes a tissue cutting device comprising a central rotatable shaft having a first end and a second end; a motor operably coupled to the first end of the central rotatable shaft, the motor comprising circuitry configured to rotate the central rotatable shaft; a moveable component configured to move along at least a portion of the length of the central rotatable shaft; and an elongated flexible cutting component having a first end and a second end, the first end of the elongated flexible cutting component secured to the moveable component and the second end of the elongated flexible cutting component secured to the central rotatable shaft in proximity to the second end of the central rotatable shaft; wherein movement of the moveable component along the at least a portion of the length of the central rotatable shaft changes a shape formed by the elongated flexible cutting component.

Also known is U.S. Patent Application Publication No. 2022/370024 A1, which discloses a device for in vivo measurements of radiopharmaceuticals used for diagnosis and monitoring of radiotherapy are presented, comprising a cannula that may comprise a measurement chamber, a radiation detector and a delivery lumen. The device is used to both deliver material to the patient (e.g., radiotracers used in radiopharmaceuticals) and measure levels and concentrations of radioactive material in, for example, the patient's blood both during and after administration of the radioactive material. In some embodiments, particles emitted by the radioactive material interact with a scintillation material, resulting in the release of light that may be transmitted, via the scintillation material and/or fiber optic material, to an optical detectors or processor for processing. In some embodiments, particle-absorbing materials may be used to limit measurements to materials within the measurement chamber or other area of interest.

Also known from the prior art is French Patent Application Publication No. 2908283 A1, which relates to a device with a portion insertable into a tubular cavity, comprising: a miniaturized gamma radiation detection probe comprising a portion insertable into a tubular cavity, which comprises at least: a scintillator crystal, which emits light in response to ionizing radiation, preferably gamma radiation, and which has a volume of less than 30 mm³, the maximum cross-sectional area of the probe being less than 10 mm², an optical fiber collecting at least some of the light emitted by the scintillation material, and its transmission to a photoelectric converter, a photoelectric converter to which the crystal is connected via the optical fiber, which collects and transmits at least some of the light emitted by the scintillator crystal to the photoelectric converter, and optical means for viewing the cavity, insertable therein.

U.S. Patent Application Publication No. 2017/131311 A1 describes a system for analysis of biopsy samples, which comprises a tissue sample transport mechanism linking a biopsy sample excision tool to a tissue sample holder disposed in a staging area of an analysis unit. The tissue sample is automatically transported from the excision tool to the specimen holder, where the tissue sample is analyzed in the staging area of the analysis unit. The transport mechanism may comprise tubing and a vacuum source. The tissue sample holder may be configured to slow or temporarily stop a tissue sample for individual analysis, or collect multiple tissue samples for analysis as a group. A tissue sample sorting mechanism may be employed that allows separation of specimens that can be correlated to the analysis.

International Patent Application Publication No. WO2018102713 A2 describes a biopsy device comprising a body, a needle, a cutter, a tissue sample holder and a gate assembly. The needle extends distally from the body. The cutter is longitudinally translatable relative to the needle and defines a cutter lumen. The tissue sample holder is coupled proximally relative to the body. The cutter lumen of the cutter defines at least a portion of a fluid conduit extending between the cutter and the tissue sample holder. The gate assembly is configured to selectively arrest movement of a tissue sample holder within the fluid conduit between the cutter and the tissue sample holder.

The prior art solutions are not entirely satisfactory, as the collection receptacle contains a mixture of all the tissue removed after the procedure, including healthy tissue, which complicates subsequent analysis work and prevents concentration on the study of pathological tissue. In addition, the management of radioactive waste by hospitals is highly regulated. Indeed, certain pathologies, such as neurological pathologies, entail a risk and require that all waste used for treatment be destroyed: The waste is then tracked, stored, and sent to an identified waste area. Mixing marked and unmarked tissue increases the volume of special waste requiring costly treatment for disposal.

The prior art solutions make it possible to quantitatively monitor in real time the usefulness of tumor excision of tissue detected by the fluorescent or radioactive marker, but not to correlate count data with other information for more sensitive tracer detection by isolating detection from the “noise” of the surgical wound.

The solutions proposed by U.S. Patent Publication Nos. 2012/283563 A1 or 2016/100852 A1 do not provide a solution, as they can detect the passage of a biological material, but cannot discern whether it is cancerous tissue or not.

### BRIEF DESCRIPTION OF THE DRAWINGS

The present disclosure will be better understood upon reading the following description, which concerns a non-limiting exemplary embodiment that is shown by the appended drawings, in which:

FIG. 1 shows a schematic view of an example of a collection system according to the present disclosure;

FIG. 2 shows a schematic view of a variant embodiment of the collection system according to the present disclosure;

FIG. 3 shows a schematic view in longitudinal cross-section of a detection sleeve for a collection system according to the present disclosure; and

FIG. 4 shows a schematic view in transverse cross-section of a detection sleeve for a collection system according to the present disclosure.

### DETAILED DESCRIPTION

The system according to the present disclosure includes:
an excision tool (100);
a suction tube (200), preferentially disposable;
a set of receptacles (300);
a vacuum source (400), possibly connected via a connecting branch (325) to the hospital vacuum source;
a computer terminal (500); and
a detection sleeve (600) surrounding the suction tube (200).

The following description specifies the features of some of the system components. It should be noted that each component can be developed and used independently of the others, and that it should not be assumed that they are all necessarily combined with one another.

The general principle of the present disclosure involves providing one or more detectors on the suction tube (200) connecting the excision tool (100) to the receptacles (300) for the aspirated products, in order to characterize in real time, during suction, within the suction tube (200), the presence and/or nature of the sample taken through the tube, in order to control one or more functions such as routing to a particular receptacle in the set (300) or emitting a sound or light signal in the event of excessive aspiration of unmarked samples.

### Excision Tool and Detection Head

The present disclosure is not limited to a particular type of excision tool, nor to a single soft suction solution, but can be implemented with any type of excision or soft suction tool that sends samples through a hose.

In one variant, the excision tool (100) includes a head (110) (preferably disposable) formed by a tubular element through which a suction channel passes, with several possible geometries that can be modified according to the operating cavity, sensitivity, or resolution required.

This head (110) is supplemented by a photodetection system (120), itself coupled to a computer terminal (500) driven by data control, acquisition, and visualization software.

The detection head (110) measures the concentration of R particles in the tissue in the vicinity of the suction mouthpiece. The signal thus received is transformed by the photodetection system into an electrical signal; this electrical signal is transmitted to the data acquisition module. The data is acquired, processed, and displayed using computer software on a terminal (500).

The detection and excision head is connected to an optical fiber equipped with a scintillator crystal at one end. The head can be inserted into the tubular cavity and moved within it, to detect the presence of any gamma radiation source, corresponding to potentially cancerous tissue that has reacted with the biomarker. The other end of the optical fiber is connected to a photoelectric converter. This photoelectric converter is most often used. The optical fiber is a means of collecting and transmitting the light emitted by the crystal. The optical fiber can have an inorganic core, e.g., silica, or a polymer core, e.g., PMMA. The part of the light emitted by the crystal that is collected by the optical fiber is transmitted by the optical fiber to the photoelectric converter, which converts it into an electrical signal and, in particular, into electrical pulses. Advantageously, the coupling between the optical fiber and the crystal ensures the collection of more than 5% of the scintillation photons emitted per event, and preferably more than 10%. To optimize the light collected, the optical fiber is preferably connected to a face of the crystal in a connection section, and the ratio between the surface area of the crystal to which the fiber is connected and the surface area of the connection section is preferably close to 1, and preferably less than or equal to 5. The optical fiber is flexible, so it can flow and adapt to the shape of the cavity in which it will be inserted. Depending on the application, it is possible to use a very long fiber, and, in particular, a length greater than or equal to 10 m. Advantageously, the core of the fiber has a diameter of preferably less than 1.5 mm, and the total fiber (core+cladding) has a diameter of preferably less than 3 mm. In a preferred embodiment, the entire insertable portion can be bent with a radius of curvature of less than 5 cm, preferably less than 2 cm. To obtain a bending radius of less than 1.5 cm with silica-core fibers, several fibers can be coupled to the same crystal. Advantageously, the insertable portion has a maximum cross-section (taken transversely to the longitudinal axis of the probe) of between 0.1 and 10 mm² and preferably between 0.2 and 5 mm². In one variant, the device further comprises means for quantifying the gamma radiation received by the crystal, by analyzing the electrical signals emitted by the photoelectric converter. This analysis can be performed, for example, by a counting unit. The count rate then reflects the amount of gamma radiation directly related to the distance between the radioactive emission zone and the probe. The device can also incorporate means for calculating the distance between the probe and the detected radioactive zone. Transmitted light can be converted by a photomultiplier or photodiode before analysis. In one embodiment, the signal from the photoelectric converter (which can be amplified if required) is connected to a discriminator (SCA for Single Channel Analyzer). The signal from the discriminator is then sent to a counting scale. When using two crystals, one slow and one fast, the signal from the photoelectric converter can be duplicated and redirected to two discriminators. One of the discriminators will have a threshold set for the detection of photons emitted by a fast crystal following the absorption of gamma radiation, while the other will have a threshold set for the detection of photons resulting from the detection of betas, the latter arriving diluted in time in the case of a slow scintillator. The probe according to the present disclosure can be used to detect gamma radiation from one of the isotopes chosen from: 99Tc, 123I, 18F or any other isotope emitting gamma photons with an energy greater than 50 KeV The probe according to the present disclosure is also suitable for detecting high-energy ionizing radiation, in particular, gamma radiation of energy greater than 300 KeV. Thanks to its miniaturization, the probe according to the present disclosure can be integrated or inserted as an accessory in various medical or diagnostic devices such as catheters, and, in particular, multi-channel catheters, endoscopes, telescopic instruments, and devices used during surgical procedures. An endoscope traditionally comprises optical means for viewing the cavity wherein it is inserted, as well as an operating channel and/or an insertion channel. The viewing means can be one or more optical fibers connected to an optoelectronic converter to obtain an image of the cavity. It is also possible to use a miniaturized camera positioned at the distal end of the endoscope. The very low weight associated with the flexible shape of the optical fiber, which allows the signal emitted by the scintillator crystal to be offset, means that the probe according to the present disclosure can be used directly in standard endoscopic channels. The optical viewing means and the gamma radiation detection probe are positioned in a guide tube, which may be compartmentalized. For example, the probe can be inserted directly into the endoscope's insertion tube or operating channel, with the scintillating crystal positioned at the distal end of the endoscope. The coupling of traditional optical detection techniques with the probe according to the present disclosure is particularly advantageous. Firstly, the probe detects the radioactive isotopes that accumulate in tumors, thus strengthening the optical diagnosis of visible tumors. In addition, the probe according to the present disclosure can detect tumors present deep in the tissue and therefore invisible to direct observation. Detecting a marking by ionizing radiation, thanks to the probe according to the present disclosure, offers an important supplement to existing optical endoscopic systems: Detection of small lesions, detection of non-surface areas (1 to 5 cm beyond the walls and not accessible optically), and more generally, detection in highly contrasted areas where optical analysis remains difficult, etc. This technique has great potential and a wide range of medical applications, which will evolve and grow with the development of increasingly specific probes and/or radiopharmaceuticals. The following examples show the present disclosure. Example 1: The detector is a rectangular crystal of LYSO: Ce3+ (Fibercryst SAS, France at 0.5% Ce), size 3*1*1 mm, polished on all sides. The optical fiber is a Thorlabs BFH37-800 fiber with an 800 mm silica core and a numerical aperture of 0.37. The total diameter of the fiber is 1.4 mm. The fiber measures 1 m and is polished at both ends. The crystal is bonded to one side of the fiber using a two-component epoxy adhesive (Geofix from ESCIL) that is transparent to the light emitted by the crystal. The probe head is then painted with white acrylic paint, and the whole probe is painted with black acrylic paint. The output end of the probe is placed on a Hamamatsu R647 photomultiplier. The signal from the photomultiplier is sent to a preamplifier and then to a shaping amplifier, before being fed back to a pulse counter, also known as a stroke counter. The probe is then placed in contact with a radioactive source of 137Cs (gamma emitter 662 KeV) with an activity of 20 KBq. The geometry of the source can be likened to a 3 mm diameter disc. The probe is then retracted parallel to the disc axis in 0.25 mm steps, and the number of strokes measured is integrated over a period of one second.

### Detection Head Structure

The detection head (120) is composed of a set of detection elements arranged circularly around an excision tool (110). Scintillation light from the fibered detection elements is directly collected and detected by a photodetection system (detection ring), consisting of silicon photomultipliers. The electrical signals generated by each SiPM are routed to an electronic acquisition system that quantifies the spectrum and count rate of beta particles detected on each fibered element.

Each of the head's detection elements is made up of two parts: A clear optical fiber, topped by a scintillating optical fiber. The scintillating optical fiber detects radioactive emissions (γ or β). When a γ or β particle interacts with the scintillating optical fiber, a spray of scintillation photons is created. The clear optical fiber then acts as a light guide, directing scintillation photons away from the surgical wound.

After its journey through tissue, a β+ particle can be annihilated by encountering an electron. Such annihilation produces two 511 keV γ photons, which, if detected, disrupt the direct measurement of beta particles. Because of their long mean path, the photons detected may come from non-specific accumulation zones of the radiotracer, located at a distance from the region of interest. This background noise must therefore be rejected to ensure the reliability of the measurement.

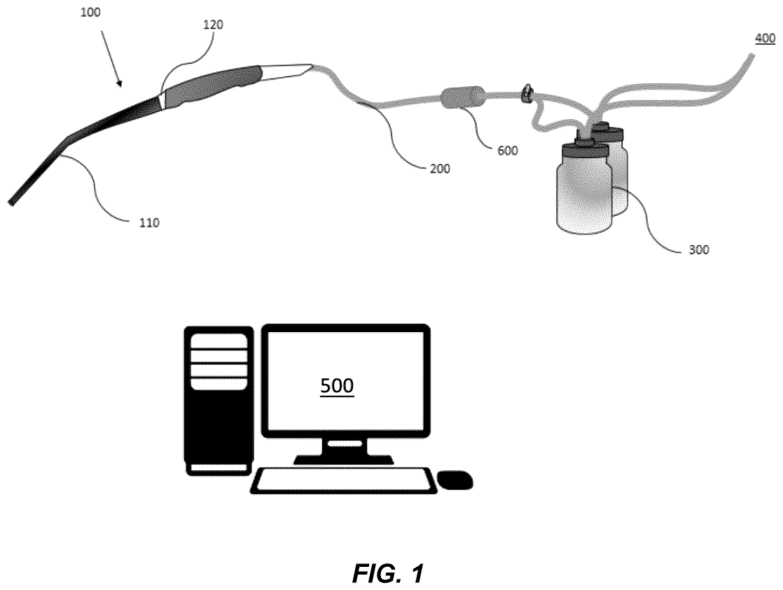

To do this, the flux of photons reaching the probe is evaluated by placing detection elements sensitive only to γ radiation. These detection elements are stainless steel-shielded optical fibers, known as “control” fibers, as opposed to unshielded fibers, known as “signal” fibers (FIG. 1). The signal from the control fibers is subtracted from the one from the signal fibers, leaving only the pure β signal.

The thickness of the scintillating parts of the “signal” fibers is optimized to achieve the best compromise in terms of maximum positron energy deposition and minimum gamma background contamination. The length of the scintillating part of the “control” fibers is defined so as to reduce statistical fluctuations in gamma signal measurement, while minimizing the subtraction method's dependence on spatial variations in background noise.

To optimize light collection by each detection element, it was decided to add an optical coating to each of the fibers, covering them with a thin layer of reflective paint that retro-guides scintillation photons emitted in the opposite direction through the clear fiber. This coating improves probe sensitivity.

The choice of using optical fibers as the probe's detection element is explained by the fact that they are both suitable for positron detection, while being relatively insensitive to γ background noise.

Moreover, since the fibers can come in a variety of shapes and sizes, they provide flexibility when it comes to designing the geometry of the detection head. So, depending on the clinical context, the pathology being treated, the shape of the surgical cavity, the excision tool chosen, etc., multiple head shapes can be proposed.

From a clinical point of view, the optical fibers also guarantee patient and surgeon safety: They are electrically inert, which means they can be introduced into the surgical wound and coupled with an excision tool. Finally, they are compact, making it possible to design a miniaturized probe.

The choice of a single-use detection head simplifies the post-operative tool sterilization procedures inherent in surgical practice, and enables the device to retain good sensitivity. An alternative approach would be to protect the probe with a plastic cover. The addition of such a cover would reduce the performance of the detection head.

The detection head is made up of plastic optical fibers and a metal part. The metal part could presumably be recovered and reused for the same purpose. As optical fibers are made of plastic, a study could be carried out into the possibility of recycling them.

The principle behind the use of such an excision tool is as follows: Once the main visible part of the tumor has been removed and radiolabeled, the surgeon introduces the probe into the surgical wound. The surgeon first removes the visible part of the tumor using an excision tool, such as an ultrasonic aspirator.

A β-emitting radiotracer is then injected intravenously during the operation, 30 to 60 minutes before the tumor margins are checked, to minimize radioactive contact with operating room staff.

The β-particle concentration is then measured using the detection head to locate tumor residues and remove and aspirate them.

Local measurement of the concentration of the radioactive tracer around the excision site makes it possible to locate any tumor residues. The sensor probe is capable of speeding up the process of searching for cancerous tissue, and enhancing the quality of the surgical procedure by enabling the surgeon to define tumor resection margins more precisely and in real time, offering improved comfort and safety.

Surgeons can therefore remove tumor tissue more precisely, while preserving the surrounding healthy tissue, thereby further preserving the affected organs. This less invasive, more conservative surgery enables patients to recover more quickly and maintain a better quality of life, while limiting the number of recurrences.

When using a detection head (110), the surgeon can choose between two display modes to receive real-time information on the location of tumor tissue: A “counter” mode and an “imager” mode. The counter mode provides the count rate β in the set of optical fibers of which the detection head is composed. The numerical value is displayed on the terminal's screen and is complemented by an audible signal with a frequency proportional to the count rate, enabling exploration to be carried out without taking one's eyes off the surgical wound. In imager mode, each optical fiber is represented visually on the screen; the count rate for each fiber is specified, accompanied by a color whose intensity is proportional to the indicated value. This image can be presented on a dedicated screen or directly on the microscope used by the surgeon, for example.

### On-Line Characterization of Tissue Samples Passing Through the Reference Zone

The present disclosure differs from the prior art more particularly in that the flow through the suction tube (200) detects the passage of marked samples prior to transfer to a receptacle (300). This detection is performed by a detection sleeve (600) located in a reference zone of the suction tube, at a known distance from the opening into the receptacle (300).

Tissue characterization, and, in particular, the presence or absence of a marker, is performed by a sleeve (600) comprising one or more sensors adapted to the traceability mode provided for the excision system.

This detection sleeve (600) passing through the suction tube (200) is arranged on a reference zone of the tube (200), determined by a distance D₁ from the downstream end of the tube (200) and a distance D₂ from the upstream end of the tube (200). Detection can be carried out by simple light barriers or by cameras, or by radiation or particle counters.

Knowing the distance D₁ and the average speed of movement of the samples in the suction tube makes it possible to determine the time offset of passage through an orientation valve (230) directing the marked samples to a first receptacle (310) via a branch (315), or to a waste receptacle (320) via another branch (325).

Both receptacles are connected to the vacuum source (400).

Information from the sleeve (600) is used to control the valve (230) depending on whether or not a marker is detected.

### Processing of the Data.

The sleeve (600) includes one or more sensors for detecting the passage of a material, or for detecting the passage of a marked sample. These sensors include a microcontroller for processing the electrical signals generated by detection. These signals are then processed, comprising some or all of the following functions:
Sampling and coding signals to provide a digital data frame;
Timestamping the data frame or data;
Filtering the data to transmit only data of interest; and
Adding incremental identifier to data.

This data is then used by an application on the terminal (500) to provide functionalities such as:
Sample routing to one of the receptacles (300) depending on the characterization of the sample in question;
Counting the number of marked samples;
Correlating with other information, such as suction pressure or suction activation, or the image acquired by the detection head, with a time shift if necessary; and
Activating a sound or light signal.

### Receptacles

Alternatively, the set of receptacles (300) comprises a barrel made up of a number of receptacles that are moved after each sample transfer. In this case, the information provided by the sleeve (600) is time-stamped and stored in a table in relation to the receptacle wherein the sample, characterized during passage through the reference zone, has been transferred.

In one variant, the receptacles include a PBS precision balance providing real-time information on the mass of samples collected.

Receptacles can be fitted with individual radioactivity sensors.

They can also be refrigerated to improve preservation of harvested tissue, or contain a solution for fixing harvested tissue, such as formaldehyde.

### Radiation Protection of the Set of Receptacles

In a preferred embodiment, the receptacles (310, 320) are enclosed in a confined radiation protection enclosure. This confined enclosure advantageously features space to house the patient's urinary catheter (450), and thus confines all sources of radiation from the patient in a radiation protection enclosure (800).

The enclosure consists of a box whose walls include lead sheets, typically sheets with a total thickness suitable for absorbing gamma radiation of energy below 1 MeV, designed to confine the gamma radiation emitted by the samples and by the patient's urine. The box has a housing for a urine bag, as well as a housing for the set of sample receptacles (300). Passages are provided for the urine bag conduits and the sample transfer tube (200), as well as the tube for connection to the hospital vacuum source, to pass through. Alternatively, some passages are replaced by fluidic connectors.

The radiation-safe receptacle (310) will preferentially feature separate compartments, each equipped with a radiation sensor (360), to accommodate selected samples, rejected samples and, where applicable, patient fluid receptacles. The receptacle will preferably house the selected samples, the rejected tissue collection (with a specific sensor) and the urine bag with a sensor (350) and volume indication (the urine bag will not be visible through the shielded containment and it is important for the surgeon and anesthetist to know the volume, color (presence of blood, for example), concentration, etc.).

### Sensors

Various types of sensor can be used to characterize the sample, in particular, a light barrier formed by a light source oriented transversely to the axis of the tube (200) associated with a photodetector or an array of photodetectors, or a camera for counting samples and optionally for imaging the sample section, and/or a photodetector detecting the luminescence of a sample marked with a phosphorescent marker, or a photodetector associated with an excitation source detecting the luminescence of a sample marked with a fluorescent marker, or a detector capable of counting the radioactivity of a sample labeled with a radioactive tracer.

The valve (230) can be controlled automatically on the basis of signals supplied by the sleeve (600), or manually by the surgeon; in the latter case, the sleeve (600) controls the activation of a sound and/or light signal on detection of a marked sample, and the surgeon decides on perception of this signal whether the sample under consideration is to be retained or rejected, possibly taking into account other criteria arising from the operation in progress.

The sleeve (600) can also be connected to a mass spectrometer to characterize the presence or absence of certain proteins or exogenous tracers (such as glucose, dopamine, etc.) or a biochemical signature of a substance of interest.

### Radioactivity-Sensitive Sensor

In a preferred embodiment, a sensor counts the radioactivity of the aspirated samples. To this end, the reference zone of the sleeve (600) comprises an inner tubular segment (610) consisting of a scintillator dedicated to the detection of beta particles. This inner tubular segment (610) is surrounded by photodetectors (630) wired on a printed circuit board (620) and covered with a reflective coating (615) (except where the photodetectors are coupled). This first scintillator (610) is surrounded by a second tubular segment (640), coaxial with the first, and made up of a scintillator dedicated to gamma radiation detection. It is covered by a reflective coating (645) and equipped with photodetectors (660) wired on a printed circuit.

The detection zone thus consists of two concentric scintillators (610, 640). Photodetectors (of the Silicon PhotoMultiplier or SiPM type) are coupled to the scintillators, on their outer face and/or at their end.

The internal beta scintillator (610) is a ring-shaped organic scintillator that detects beta particles and gamma radiation. It is cylindrical on the inside and faceted on the outside for optimum contact with the silicon photomultipliers (630) responsible for collecting the signal.

The external scintillator, called the “gamma scintillator” (640), is an inorganic scintillator, also annular, denser than the internal scintillator (610), which is superimposed on the latter and detects only gamma rays—the short mean path of beta particles emitted by the sample does not allow them to reach the gamma scintillator (640).

Using the data collected by the two scintillators (610, 640) via the photodetectors (630, 660), it is possible to deduce the beta or gamma count rate of the sample proportional to the amount of radioactive tracer present in the sample, using a coincidence method.

The system thus makes it possible to measure:
Beta count rate (with gamma signal correction); and
Gamma count rate only.

A visual or audible safety alert is triggered when a sample passes through the detection sleeve without causing beta particle detection. In this case, the sample has been taken from an unmarked and therefore non-cancerous area, and the operator is warned by the alert to avoid further unnecessary sampling.

### Additional Sensors

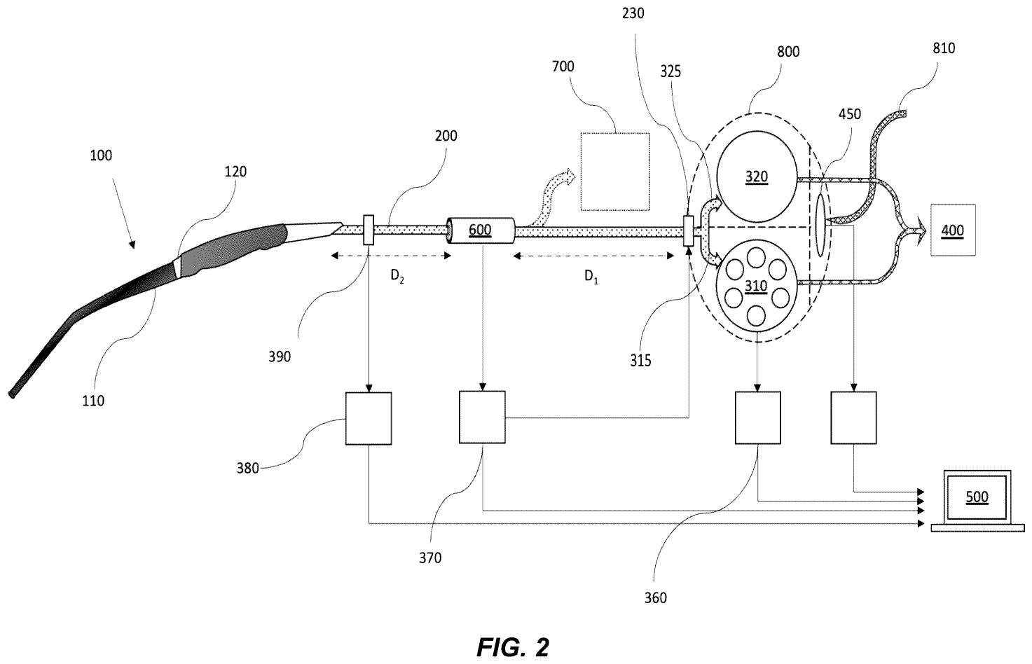

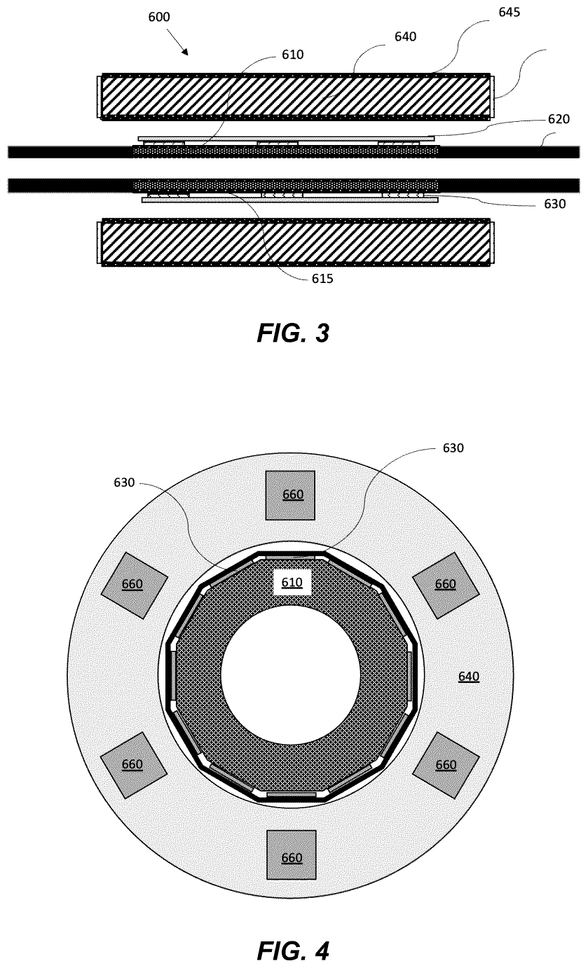

FIG. 2 shows an example of a design with additional sensors.

A light barrier (390) formed by a light source arranged to form a beam passing transversely through the tube (200) and a photodetector delivers a signal that varies when a sample passes through the beam field of the light barrier (390). A microcontroller (380) processes the electrical signal delivered by the light barrier (390) to deliver time-stamped information on the passage of the sample, or with an identifier correlated with an identifier or timestamp of the image of the sampling zone acquired with the excision tool.

A sensor (370) detects the passage of the sample out of the sleeve (600) and provides a time stamp of this passage. Activation of the sensor can, depending on the detection result delivered by the sleeve (600), trigger a safety alert (sound or light signal) and/or control the valve (230). The two detection zones (390, 600) are spaced apart by a distance D₂, and by measuring the time elapsed between them, it is possible to determine the speed of movement of the sample in the tube (200) and characterize any anomalies by comparison with the nominal speed of movement.

Another light barrier (230) also provides information on the time elapsed between the passage of the sample through the sleeve and the travel of the pipe section of length D₁, and provides an alert in the event of drift in relation to a reference time.

The system can also be equipped with an additional analysis means (700) connected to the detection sleeve (600), for example, a mass spectrometer, or a fluorescence counter.

### Sequencing of Samples According to A Non-Limiting Example

The surgeon controls tissue aspiration: the head of the excision tool in contact with the patient's tissue aspirates tissue samples, which are then drawn through the tube (200) to the set of receptacles (300).

The samples are drawn by this suction through the tube (200) and pass into the reference zone where the detection sleeve checks the presence or absence of markers in the sample passing through it at a given time t, enabling it to be directed toward a waste receptacle or a receptacle for subsequent analysis.

According to a particular embodiment, a pressure switch-type sensor detects the closure of the hole and, thus the start of a sampling sequence, and the state of this sensor is time-stamped and recorded, along with the image of the sampling zone in the surgical wound supplied by the detection head (120), which is also recorded in time-stamped form.

The signals supplied by the sleeve (600) are also time-stamped.

The resynchronization of information takes into account the average times traveled by a sample in the tube (200) in the length D₂ between the excision tool and the sleeve (600) and in the length D₁ between the receptacle (300) and the sleeve (600).

This information is used to provide additional information about the tissue sampled and collected at a given time. By providing a barrel with a plurality of receptacles, each tissue sequence removed is collected in a specific receptacle, associated with a reference time, which may be used to associate it with the image supplied by the excision tool's imager and the information supplied by the sleeve (600).

1. A system for sampling biological tissue from a zone of interest receiving a biomarker prior to or simultaneously with surgery, the system comprising:
an excision head connected to a receptacle for the tissue samples by a suction transfer tube;
a detection sleeve surrounding a reference zone of the suction transfer tube, the detection sleeve comprising at least one sensor sensitive to the biomarker; and
an electronic circuit delivering an electrical signal representative of a presence or absence of a marker when a tissue sample passes through the reference zone.

2. The system of claim 1, wherein the electrical signal controls transfer of a tissue sample that has just passed through the reference zone to the receptacle in the event of detection of a marker, or to another receptacle by default.

3. The system of claim 1, further comprising a transient suction actuator delivering a time-stamped electrical signal, wherein the signal representative of the presence of a marker being time-stamped.

4. The system of claim 1, wherein the sensor is formed by an annular organic scintillator, which detects beta particles and gamma rays, the scintillator being associated with a photodetector.

5. The system of claim 1, wherein an annular organic scintillator is surrounded by an annular inorganic gamma scintillator, which detects only gamma rays, the inorganic gamma scintillator being associated with a second photodetector.

6. The system of claim 1, further comprising a valve directing the sampled tissue toward a collection receptacle or toward a waste receptacle depending on information delivered by the detection sleeve.

7. The system of claim 1, wherein the receptacle comprises a barrel including a plurality of collection units.

8. The system of claim 1, further comprising a pressure switch detecting activation of sampling by the excision head.

9. The system of claim 1, further comprising an enclosure having walls comprising lead, sheets for containment of the receptacle.

10. The system of claim 9, wherein the enclosure comprises a plurality of housings each comprising a radiation sensor.

11. The system of claim 9, wherein the enclosure further comprises a housing for receiving a urine bag.

12. The system of claim 1, wherein the electronic circuit further controls a safety alert in an event of non-detection of a biomarker.

13. The system of claim 1, further comprising means for real-time histological and biochemical analysis of the tissue
